(19) United States
(12) Patent Application Publication
(10) Pub. No.: US 2011/0263844 A1
(43) Pub. Date: Oct. 27, 2011

(54) METHOD FOR PREPARING MACROCYCLES

(75) Inventors: Chutian Shu, Danbury, CT (US); Chris Hugh Senanayake, Brookfield, CT (US); Zhulin Tan, Danbury, CT (US); Nathan Yee, Danbury, CT (US); Xingzhong Zeng, New Milford, CT (US)

(73) Assignee: BOEHRINGER INGELHEIM INTERNATIONAL GMBH, Ingelheim am Rhein (DE)

(21) Appl. No.: 12/991,238

(22) PCT Filed: May 5, 2009
(86) PCT No.: PCT/US09/42773

§ 371 Date: Jun. 6, 2011

Related U.S. Application Data
(60) Provisional application No. 61/051,809, filed on May 9, 2008.

(51) Int. Cl. C07D 487/04; C07D 403/12

(52) U.S. Cl. 540/460; 548/465

(57) ABSTRACT

The present invention is directed to a method for the preparation of macrocyclic compounds of formula (I),

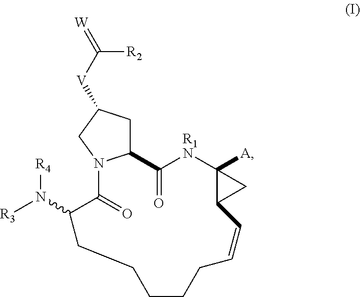

comprising the step of cyclizing a diene of formula (II),

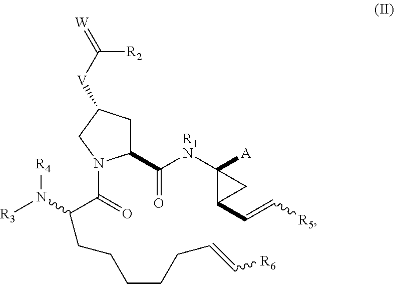

in the presence of a catalyst, wherein R₁-R₆, A, W and V are as defined herein. The present invention is also directed to intermediate compounds of formula II.

### TECHNICAL FIELD

The invention relates to an improved process for the preparation of certain macrocyclic compounds useful as agents for the treatment of hepatitis C viral (HCV) infections, or as intermediates useful in preparing such agents.

### BACKGROUND INFORMATION

The macrocyclic compounds of the following formula and methods for their preparation are known from: Tsantrizos et al., U.S. Pat. No. 6,608,027 B1; Llinas Brunet et al, U.S. Pat. No. 7,119,072; Llinas Brunet et al, U.S. Pat. No. 7,504,378; Llinas Brunet et al, U.S. Application Publication No. 2005/0080005 A1; Brandenburg et al., U.S. Pat. No. 7,148,347 and Samstag et al., U.S. Application Publication No. 2004/0248779 A1:

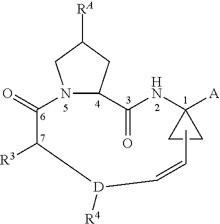

wherein
R^{A} is OH, O-PG, where PG is a protecting group, or —OSO₂—R²⁷, wherein R²⁷ is selected from phenyl, p-tolyl, p-bromophenyl, p-nitrophenyl, methyl, trifluoromethyl, perfluorobutyl and 2,2,2-trifluoroethyl;
or a group of formula II

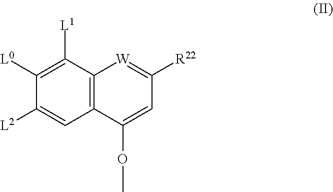

### W is CH or N,

L⁰ is H, halo, C₁₋₆ alkyl, C₃₋₆ cycloalkyl, C₁₋₆ haloalkyl, C₁₋₆ alkoxy, C₃₋₆ cycloalkoxy, hydroxy, or N(R²³)₂,
wherein each R²³ is independently H, C₁₋₆ alkyl or C₃₋₆ cycloalkyl;
L¹, L² are each independently H, halogen, C₁₋₄ alkyl, —O—C₁₋₄ alkyl, or —S—C₁₋₄ alkyl (the sulfur being in any oxidized state); or

### L⁰ and L¹ or

L⁰ and L² may be covalently bonded to form together with the two C-atoms to which they are linked a 4-, 5- or 6-membered carbocyclic ring wherein one or two (in the case of a 5- or 6-membered ring) —CH₂— groups not being directly bonded to each other, may be replaced each independently by —O— or NR^{a} wherein R^{a} is H or C₁₋₄alkyl, and wherein said ring is optionally mono- or di-substituted with C₁₋₄ alkyl;
R²² is H, halo, C₁₋₆ alkyl, C₃₋₆ cycloalkyl, C₁₋₆ haloalkyl, C₁₋₆ thioalkyl, C₁₋₆ alkoxy, C₃₋₆ cycloalkoxy, C₂₋₇ alkoxyalkyl, C₃₋₆ cycloalkyl, C_{6 or} C₁₀ aryl or Het, wherein Het is a five-, six-, or seven-membered saturated or unsaturated heterocycle containing from one to four heteroatoms selected from nitrogen, oxygen and sulfur; said cycloalkyl, aryl or Het being substituted with R²⁴,
wherein R²⁴ is H, halo, C₁₋₆ alkyl, C₃₋₆ cycloalkyl, C₁₋₆ alkoxy, C₃₋₆ cycloalkoxy, NO₂, N(R²⁵)₂, NH—C(O)—R²⁵; or NH—C(O)—NH—R²⁵, wherein each R²⁵ is independently: H, C₁₋₆ alkyl or C₃₋₆ cycloalkyl;
or R²⁴ is NH—C(O)—OR²⁶ wherein R²⁶ is C₁₋₆ alkyl or C₃₋₆ cycloalkyl;
R³ is hydroxy, NH₂, or a group of formula —NH—R⁹, wherein R⁹ is C_{6 or 10} aryl, heteroaryl, —C(O)—R²⁰, —C(O)—NHR²⁰ or —C(O)—OR²⁰, wherein R²⁰ is C₁₋₆ alkyl or C₃₋₆ cycloalkyl;
D is a 3 to 7 atom saturated alkylene chain optionally containing one to three heteroatoms independently selected from: O, S or N—R²⁷, wherein R²⁷ is H, C₁₋₆alkyl, C₃₋₆cycloalkyl or C(O)R²⁸, wherein R²⁸ is C₁₋₆alkyl, C₃₋₆cycloalkyl or C_{6 or 10} aryl;
R⁴ is H, or from one to three substituents at any carbon atom of said chain D, said substituent independently selected from the group consisting of: C₁₋₆ alkyl, C₁₋₆ haloalkyl, C₁₋₆ alkoxy, hydroxy, halo, amino, oxo, thio, or C₁₋₆ thioalkyl; and

A is an amide of formula —C(O)—NH—R¹¹, wherein R¹¹ is selected from the group consisting of: C₁₋₈ alkyl, C₃₋₆ cycloalkyl, C_{6 or 10} aryl, C₇₋₁₆ aralkyl, or SO₂R^{5A} wherein R^{5A} is C₁₋₈ alkyl, C₃₋₇ cycloalkyl, C₁₋₆ alkyl-C₃₋₇ cycloalkyl;

or A is a carboxylic acid or a pharmaceutically acceptable salt or ester thereof.

International Publication No. WO 2005/037214 discloses similar compounds.

The compounds disclosed in the above-mentioned patent documents as being active agents for the treatment of hepatitis C viral (HCV) infections, or as intermediates useful for the preparation of such anti-HCV agents as described therein, and are prepared therein via ring-closing metathesis of an acyclic diolefin using ruthenium-based catalysts in a suitable organic solvent. The disadvantages of the previously reported approaches to the compound via ring-closing metathesis include long reaction time, high catalyst loading, moderate yields, and the need to use lower concentrations of the diene substrate to obtain optimum results. Thus, there is a continuing need in the art to develop improved processes for obtaining the macrocyclic compounds.

### BRIEF SUMMARY OF THE INVENTION

The present invention is directed to a method for the preparation of macrocyclic compounds of formula (I),

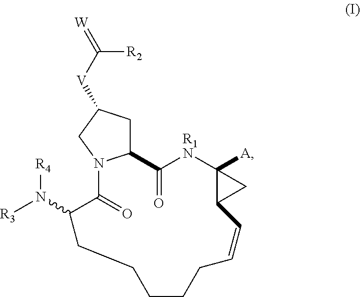

comprising the step of cyclizing a diene of formula (II),

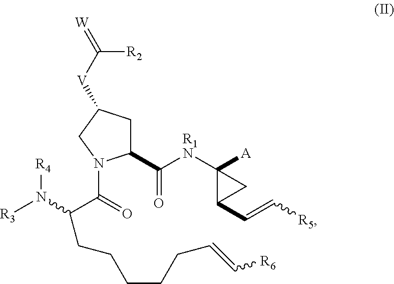

in the presence of a catalyst, wherein R₁ is an electron-withdrawing amido protecting group such as alkylcarbonyl, arylcarbonyl, alkoxycarbonyl, carbonyl, and sulfonyl, R₂ can be aryl, alkenyl, alkynyl, haloalkyl-O—, heteroaryl, heterocycloalkyl, alkoxy, aryloxy, heteroaryloxy, heterocycloalkoxy, or —NRR′, wherein R and R′ are independently selected from H, alkyl, cycloakyl, aryl, and heteroaryl, R₃ can be C(O)R⁷, C(O)OR⁷, or C(O)NR⁷R^{7′}, wherein R⁷ and R^{7′} are alkyl, cycloalkyl, or aryl, R₄ can be H, alkyl, cycloalkyl, aryl or an amino protecting group, R₅ and R₆ can independently be H, alkyl, alkenyl, aryl, or cycloalkyl.

A can be COOH, COOR⁸, CHO, CN or CON(R⁹)SO₂R¹⁰, wherein R⁸ is alkyl, aryl, heteroaryl, R⁹ is H or an amido protecting group, and R¹⁰ is alkyl, cycloalkyl, aryl, or heteroaryl, W is O and V is O, N or S, or salts thereof.

The present invention is also directed to an intermediate compound of formula II:

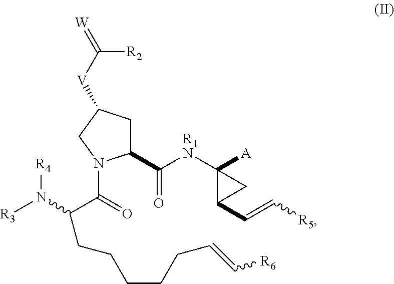

wherein R₁ is an electron-withdrawing amido protecting group, R₂ is selected from aryl, heteroaryl, and heterocycloalkyl, R₃ is C(O)R⁷, C(O)OR⁷, or C(O)NR⁷R^{7′}, wherein R⁷ and R^{7′} are independently selected from alkyl, cycloalkyl, and aryl, R₄ is H, alkyl, cycloalkyl, aryl or an amino protecting group, R₅ and R₆ are independently selected from H, alkyl, alkenyl, aryl, and cycloalkyl, A is COOH, COOR⁸, CHO, CN or CON(R⁹)SO₂R¹⁰, wherein R⁸ is alkyl, aryl, or heteroaryl, R⁹ is H or an amido protecting group, and R¹⁰ is alkyl, cycloalkyl, aryl, or heteroaryl, W is O, and V is O, N or S, or salts thereof.

### DETAILED DESCRIPTION OF THE INVENTION

The present invention is directed toward a method of synthesizing macrocyclic compounds of formula (I) from corresponding diene compounds of formula (II) in the presence of a catalyst, wherein the amide nitrogen adjacent to the cyclopropyl ring is protected by an electron withdrawing amido protecting group.

In the past, such a cyclization would require a dilute concentration of the diene, large quantities of the catalyst and long reaction times in order to obtain the desired macrocyclic compound in only modest yields. International Publication No. WO 2005/037214 discloses the need for a dilution of at least 0.01M of the diene, 25% (mol/mol) of the catalyst, and a reaction time of at least 16 hours. These conditions are not practical, especially for large-scale synthesis.

However, using the present inventive methodology, the desired macrocyclic compound of formula (I) can be synthesized from the corresponding diene in higher concentration, with less catalyst, in less time and in substantially higher yields. This allows for the large-scale production of the macrocyclic compound with more efficiency and at substantially reduced cost.

Terms not specifically defined herein should be given the meanings that would be given to them by one of ordinary skill in the art in light of the disclosure and the context. As used in the present specification, however, unless specified to the contrary, the following terms have the meaning indicated and the following conventions are adhered to.

In the groups, radicals, or moieties defined below, the number of carbon atoms is often specified preceding the group, for example, C₁₋₆ alkyl means an alkyl group or radical having 1 to 6 carbon atoms. In general, for groups comprising two or more subgroups, the last named group is the radical attachment point, for example, “thioalkyl” means a monovalent radical of the formula HS-alkyl-. Unless otherwise specified below, conventional definitions of terms control and conventional stable atom valences are presumed and achieved in all formulas and groups.

The term “alkyl” as used herein, either alone or in combination with another substituent, means acyclic, straight or branched chain alkyl substituents. Such moieties may contain up to ten carbon atoms, but preferably contain 1 to 6 carbon atoms and more preferably contain 1 to 4 carbon atoms. The term “cycloalkyl” refers to a cyclic alkyl moiety, such as for example cyclohexanyl. A cycloalkyl moiety may contain 3 to 10 carbon atoms, but preferably contains 3 to 7 carbon atoms.

The term “alkenyl” refers to branched and unbranched alkenyl groups with 2 to 6 carbon atoms and by the term “C₂₋₄-alkenyl” refers to branched and unbranched alkenyl groups with 2 to 4 carbon atoms, provided that they have at least one double bond. Alkenyl groups with 2 to 4 carbon atoms are preferred. Examples include: ethenyl or vinyl, propenyl, butenyl, pentenyl, or hexenyl. Unless stated otherwise, the definitions propenyl, butenyl, pentenyl and hexenyl include all the possible isomeric forms of the groups in question. Thus, for example, propenyl includes 1-propenyl and 2-propenyl, butenyl includes 1-, 2- and 3-butenyl, 1-methyl-1-propenyl, 1-methyl-2-propenyl etc.

By the term “alkynyl” (including those which are part of other groups) refers to branched and unbranched alkynyl groups with 2 to 6 carbon atoms and by the term “C₂₋₄-alkynyl” refers to branched and unbranched alkynyl groups with 2 to 4 carbon atoms, provided that they have at least one triple bond. Alkynyl groups with 2 to 4 carbon atoms are preferred. Examples include: ethynyl, propynyl, butynyl, pentynyl or hexynyl. Unless stated otherwise, the definitions propynyl, butynyl, pentynyl and hexynyl include all the possible isomeric forms of the groups in question. Thus for example propynyl includes 1-propynyl and 2-propynyl, butynyl includes 1,2- and 3-butynyl, 1-methyl-1-propynyl, 1-methyl-2-propynyl etc.

The term “alkoxy” as used herein, either alone or in combination with another substituent, means the substituent alkyl-O—, wherein alkyl is as defined above. Such moieties may contain up to ten carbon atoms, but preferably contain 1 to 6 carbon atoms and more preferably contain 1 to 4. Similarly, “aryloxy” means an aryl-O-group, wherein aryl is as defined herein.

The term “cycloalkoxy” as used herein, either alone or in combination with another substituent, means the substituent cycloalkyl-O—, which contains from 3 to 10 carbon atoms, and more preferably 3 to 7 carbon atoms.

The term “aryl” as used herein, either alone or in combination with another substituent, means either an aromatic monocyclic system containing 6 carbon atoms or an aromatic bicyclic system containing 10 carbon atoms. For example, aryl includes a phenyl or a naphthyl ring system.

The term “heterocycloalkyl” as used herein, either alone or in combination with another substituent, means a monovalent substituent derived by removal of a hydrogen from a five-, six-, or seven-membered saturated or unsaturated (not including aromatic) heterocycle containing carbon atoms and from one to four ring heteroatoms selected from nitrogen, oxygen and sulfur. Examples of suitable heterocycloalkyls include: tetrahydrofuran, thiophene, diazepine, isoxazole, piperidine, dioxane, morpholine, pyrimidine or

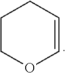

The term also includes a heterocycle as defined above fused to one or more other cyclic systems, whether a heterocycle or a carbocycle, each of which may be saturated or unsaturated. Examples include thiazolo[4,5-b]-pyridine and isoindoline. Preferably such moieties contain 1 to 9 carbon atoms.

The term “heteroaryl” as used herein precisely defines an unsaturated heterocycle for which the double bonds form an aromatic system. Suitable example of heteroaromatic “heteroaryl” systems include: quinoline, indole, pyridine,

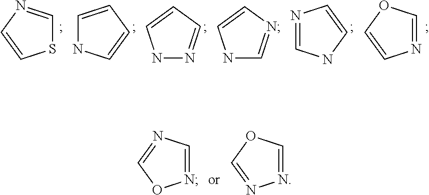

Preferably such moieties contain 1 to 9 carbon atoms.

The term “haloalkyl” refers to alkyl groups, as defined above, that is substituted with halogen atom(s), such as F, Cl, Br and I. F and Cl substituted alkyls are the preferred haloalkyl groups, for example —CF₃ and —CCl₃.

The term “carbonyl” as used herein, either alone or in combination with another substituent, means an oxo group, i.e. —C(O)—. Accordingly, an alkylcarbonyl group means alkyl-C(O)—; an arylcarbonyl group means aryl-C(O)—; and an alkoxycarbonyl group means alkyl-O—C(O)—.

The term “sulfonyl” as used herein, either alone or in combination with another substituent, means —SO₂—R, wherein R is H, alkyl, haloalkyl or aryl. Examples include —SO₂—CH₃, —SO₂—CF₃, —SO₂H and —SO₂-Ph.

The term “amido protecting group” refers to a moiety that can mask an amide functionality, but under appropriate conditions can be easily removed. One of ordinary skill in the art would be aware of numerous possibilities known in the literature, for example, Greene, Protective Groups in Organic Synthesis, 2^{nd} Ed., Wiley & Sons, 1991, ISBN: 0-471-62301-6, hereby incorporated by reference. Common examples of such groups are t-BOC and acetyl. The term “electron withdrawing amido protecting group” refers to an amido protecting group, as defined above, which draws electrons to itself more than a hydrogen atom, if it occupied the same position in a given molecule. Examples of such groups include t-BOC and acetyl.

The term “amino protecting group” refers to a moiety that can mask an amine functionality, but under appropriate conditions can be easily removed. One of ordinary skill in the art would be aware of numerous possibilities known in the literature, for example, Greene, Protective Groups in Organic Synthesis, 2^{nd} Ed., Wiley & Sons, 1991, ISBN: 0-471-62301-6. Common examples of such groups are t-BOC and acetyl.

The above-mentioned substituents, moieties, groups and functionalities can be further substituted with suitable substituents. A skilled artisan would readily be aware of which substituents would be suitable.

In general, all tautomeric forms and isomeric forms and mixtures, whether individual geometric isomers, stereoisomers, optical isomers or racemic or non-racemic mixtures of isomers, of a chemical structure or compound are intended, unless the specific stereochemistry or isomeric form is specifically indicated in the compound name or structure.

The term “pharmaceutically acceptable ester” as used herein, either alone or in combination with another substituent, means esters of the compound of formula I in which any of the carboxylic acid functions of the molecule, but preferably the carboxy terminus, is replaced by an alkoxycarbonyl function:

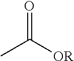

in which the R moiety of the ester is selected from alkyl (e.g. methyl, ethyl, n-propyl, t-butyl, n-butyl); alkoxyalkyl (e.g. methoxymethyl); alkoxyacyl (e.g. acetoxymethyl); aralkyl (e.g. benzyl); aryloxyalkyl (e.g. phenoxymethyl); aryl (e.g. phenyl), optionally substituted with halogen, C₁₋₄ alkyl or C₁₋₄ alkoxy. Other suitable prodrug esters are found in Design of Prodrugs, Bundgaard, H. Ed. Elsevier (1985) incorporated herewith by reference. Such pharmaceutically acceptable esters are usually hydrolyzed in vivo when injected in a mammal and transformed into the acid form of the compound of formula I.

With regard to the esters described above, unless otherwise specified, any alkyl moiety present advantageously contains 1 to 16 carbon atoms, particularly 1 to 6 carbon atoms. Any aryl moiety present in such esters advantageously comprises a phenyl group.

In particular the esters may be a C₁₋₁₆ alkyl ester, an unsubstituted benzyl ester or a benzyl ester substituted with at least one halogen, C₁₋₆ alkyl, C₁₋₆ alkoxy, nitro or trifluoromethyl.

The term “pharmaceutically acceptable salt” as used herein includes those derived from pharmaceutically acceptable bases. Examples of suitable bases include choline, ethanolamine and ethylenediamine. Na⁺, K⁺, and Ca⁺⁺ salts are also contemplated to be within the scope of the invention (also see Pharmaceutical Salts, Birge, S. M. et al., J. Pharm. Sci., (1977), 66, 1-19, incorporated herein by reference).

### General Synthetic Method:

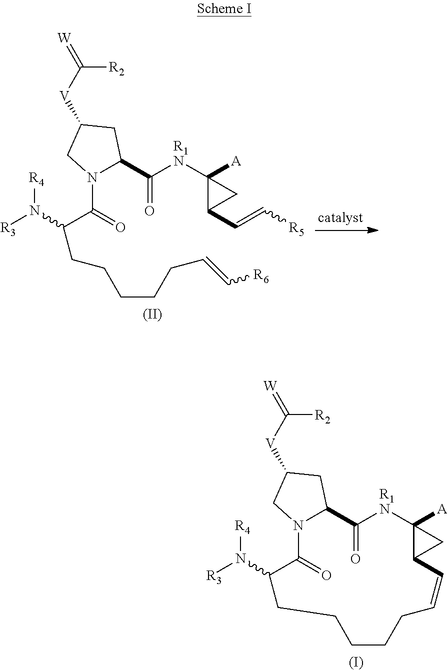

Scheme I illustrates a general synthesis of macrocyclic compounds of formula (I), wherein R₁-R₆, A, V, and W are as defined herein.

In the synthesis of a macrocyclic compound of formula (I), a diene compound of formula (II) is cyclizied in the presence of a catalyst. A skilled artisan would be aware of suitable catalysts for such a reaction. Preferred catalysts are imidazolium carbene or a saturated-imidazolium carbene based catalyst, such as Grubbs' 2^{nd} generation catalyst and Hoveyda-Grubbs' 2^{nd} generation catalyst. The most preferred catalyst is Greta catalyst, [1,3-bis-(2,4,6-trimethylphenyl)-2-imidazolidinylidene)dichloro(5-nitro-2-isopropoxyphenylmethylene)ruthenium]. In the prior art, typically catalyst loading was 25% by moles relative to the diene compound. However, when the amide nitrogen adjacent to the cyclopropyl ring is protected by an electron withdrawing protecting group, such as for example t-BOC or acetyl, then less than about 25% (mol/mol) of the catalyst is needed to obtain the desired macrocyclic compound in high yields, and more specifically only about 0.1% can be used to obtain cyclization in high yield.

Traditionally, such cyclization reaction were performed in aprotic organic solvents in high dilution, usually 0.01M. A skilled artisan would know of suitable aprotic solvents for use in this synthesis, however, toluene in preferred. When R₁ is a electron withdrawing amido protecting group, the cyclization can be performed in concentrations greater than about 0.01M while still obtaining high yields of the desired macrocyclic compound. Preferably, the concentration can be about 0.10M.

### Specific Synthetic Method:

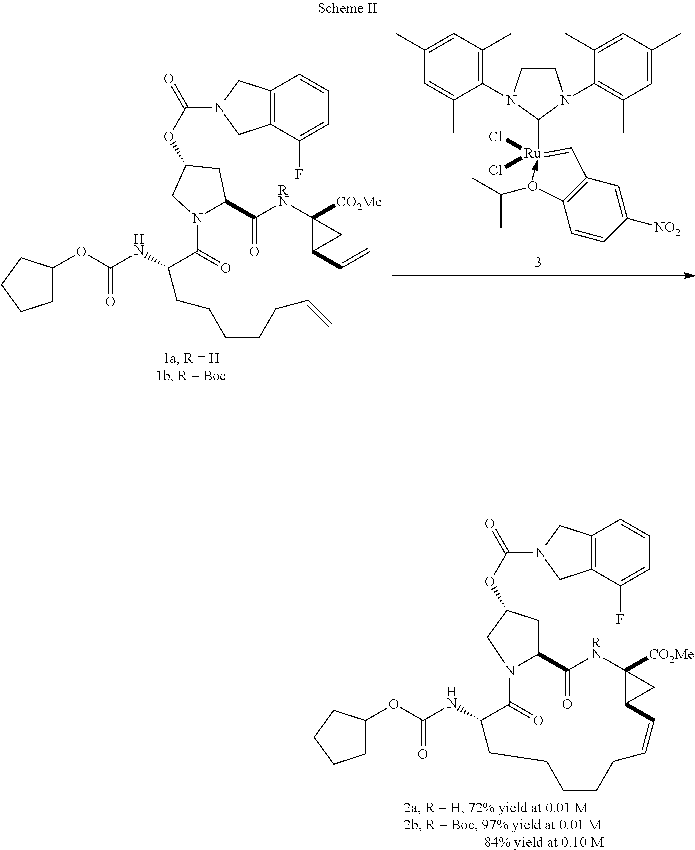

Scheme II illustrates the specific synthesis of (Z)-(1S,4R,6S,14S,18R)-14-cyclopentyloxycarbonylamino-18-(4-fluoro-1,3-dihydro-isoindole-2-carbonyloxy)-2,15-dioxo-3,16-diaza-tricyclo[14.3.0.0^{4,6}]nonadec-7-ene-3,4-dicarboxylic acid 3-(3R,5S)-5-[tert-butyl ester 4butoxycarbonyl-((1R,2R)-1-methoxycarbonyl-2-methyl ester-cyclopropyl)-aminocarbonyl]-1-((S)-2-cyclopentyloxycarbonylamino-non-8-enoyl)-pyrrolidin-3-yl ester compared to the synthesis of (Z)-(1S,4R,6S,14S,18R)-14-cyclopentyloxycarbonylamino-18-(4-fluoro-1,3-dihydro-isoindole-2-carbonyloxy)-2,15-dioxo-3,16-diaza-tricyclo[14.3.0.0^{4,6}]nonadec-7-ene-4-carboxylic acid (3R,5S)-1-((S)-2-cyclopentyloxycarbonylamino-non-8-enoyl)-5-((1R,2R)-1-methoxycarbonyl-2-methyl-cyclopropylcarbamoyl)-pyrrolidin-3-yl ester.

The corresponding diene compound, 1b, is cyclized in the presences of 1,3-bis-(2,4,6-trimethylphenyl)-2-imidazolidinylidene)dichloro(5-nitro-2-isopropoxyphenylmethylene)ruthenium to obtain the macrocylic compound, 2b, in high yield. Specifically, Scheme II shows that when R is a electron withdrawing amido protecting group, such a t-BOC, better yields of the desired product are obtained than when R is H, even when the reaction concentration is ten times greater, 0.10M.

Additionally, having an electron withdrawing amido protecting group on the nitrogen adjacent to the cyclopropyl moiety also reduces the reaction time. When R is H, the reaction typically requires about 16 hours or more, but when R is an electron withdrawing amido protecting group, such as t-BOC, then reaction is completed in less than about 16 hours, and can be complete in only about 30 minutes.

The following example is presented for illustrative purposes to provide the reader with a better understanding of the present invention and in no way should be viewed as limiting the scope of the invention.

### EXAMPLES

### Example 1

### (Z)-(1S,4R,6S,14S,18R)-14-Cyclopentyloxycarbonylamino-18-(4-fluoro-1,3-dihydro-isoindole-2-carbonyloxy)-2,15-dioxo-3,16-diaza-tricyclo[14.3.0.0^{4,6}]nonadec-7-ene-3,4-dicarboxylic acid 3-tert-butyl ester 4-methyl ester

To a three-neck flask with 1b [4-fluoro-1,3-dihydro-isoindole-2-carboxylic acid (3R,5S)-5-[tert-butoxycarbonyl-((1R,2 S)-1-methoxycarbonyl-2-vinyl-cyclopropyl)-aminocarbonyl]-1-((S)-2-cyclopentyloxycarbonylamino-non-8-enoyl)-pyrrolidin-3-yl ester] (3.9 g) in toluene (50 mL) at 110° C. was added 1,3-bis-(2,4,6-trimethylphenyl)-2-imidazolidinylidene)dichloro(5-nitro-2-isopropoxyphenylmethylene)ruthenium (12 mg in 4 mL toluene) over 30 min. The reaction was monitored by HPLC after 10 min. After the conversion reached >99%, the reaction was stopped by quenching with imidazole (50 mg) and stirred for additional 1 h at 80° C. The reaction was extracted with 1 M HCl (2×20 mL) and concentrated to give a toluene solution (20 mL) of the crude product.

1. A method for the preparation of macrocyclic compounds of formula (I), 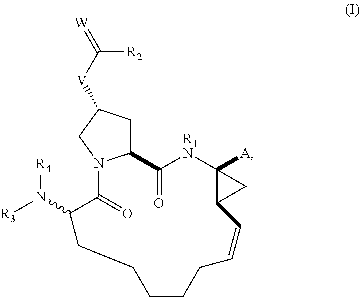
comprising the step of cyclizing a diene of formula (II), 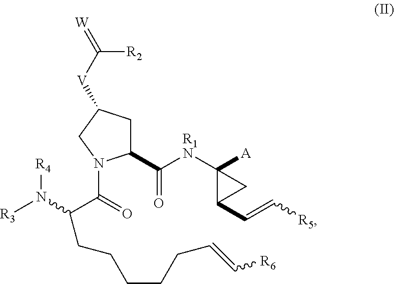
in the presence of a catalyst,
wherein:
R₁ is an electron-withdrawing amido protecting group;
R₂ is selected from aryl, alkenyl, alkynyl, haloalkyl-O—, heteroaryl, heterocycloalkyl, alkoxy, aryloxy, heteroaryloxy, heterocycloalkoxy, and —NRR′, wherein R and R′ are independently selected from H, alkyl, cycloakyl, aryl, and heteroaryl;
R₃ is C(O)R⁷, C(O)OR⁷, or C(O)NR⁷R^{7′}, wherein R⁷ and R^{7′} are independently selected from alkyl, cycloalkyl, and aryl;
R₄ is H, alkyl, cycloalkyl, aryl or an amino protecting group;
R₅ and R₆ are independently selected from H, alkyl, alkenyl, aryl, and cycloalkyl;
A is COOH, COOR⁸, CHO, CN or CON(R⁹)SO₂R¹⁰, wherein R⁸ is alkyl, aryl, or heteroaryl, R⁹ is H or an amido protecting group, and R¹⁰ is alkyl, cycloalkyl, aryl, or heteroaryl;
W is O; and
V is O, N or S;
or salts thereof.

2. The method of claim 1, wherein W and V are oxygen.

3. The method of claim 1, wherein R₄ is H or alkyl and R₃ is C(O)OR⁷.

4. The method of claim 3, wherein R⁷ is cycloalkyl.

5. The method of claim 1, wherein R₂ is heteroaryl, heterocycloalkyl, or —NRR′, wherein R and R′ are independently selected from H, alkyl, cycloakyl, aryl, and heteroaryl.

6. The method of claim 5, wherein R₂ is heterocycloalkyl.

7. The method of claim 1, wherein A is COOR⁸.

8. The method of claim 1, wherein R₅ and R₆ are H.

9. The method of claim 1, wherein R₁ is acetyl or t-BOC.

10. The method of claim 1, wherein R₁ is t-BOC.

11. The method of claim 1, wherein the concentration of the diene compound of formula (II) is greater than about 0.01M.

12. The method of claim 11, wherein the concentration of the diene compound of formula (II) is about 0.10M.

13. The method of claim 1, wherein the catalyst is 1,3-bis-(2,4,6-trimethylphenyl)-2-imidazolidinylidene)dichloro(5-nitro-2-isopropoxyphenylmethylene)ruthenium.

14. The method of claim 1, wherein the time required to convert 99% of the diene compound of formula (II) into the macrocyclic compound of formula (I) is less than about 16 hours.

15. The method of claim 1, wherein the time required to convert 99% of the diene compound of formula (II) into the macrocyclic compound of formula (I) is about 0.5 hours.

16. The method of claim 1, wherein the catalyst is present in an amount of less than about 25% (mol/mol).

17. The method of claim 1, wherein the catalyst is present in an amount of about 0.1% (mol/mol).

18. A compound of formula II: 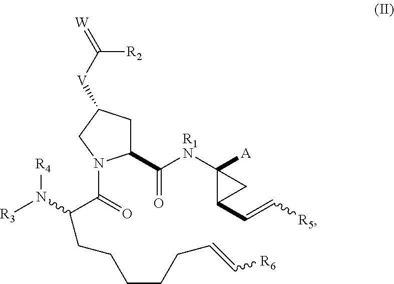
wherein:
R₁ is an electron-withdrawing amido protecting group;
R₂ is selected from aryl, heteroaryl, and heterocycloalkyl;
R₃ is C(O)R⁷, C(O)OR⁷, or C(O)NR⁷R^{7′}, wherein R⁷ and R^{7′} are independently selected from alkyl, cycloalkyl, and aryl;
R₄ is H, alkyl, cycloalkyl, aryl or an amino protecting group;
R₅ and R₆ are independently selected from H, alkyl, alkenyl, aryl, and cycloalkyl;
A is COOH, COOR⁸, CHO, CN or CON(R⁹)SO₂R¹⁰, wherein R⁸ is alkyl, aryl, or heteroaryl, R⁹ is H or an amido protecting group, and R¹⁰ is alkyl, cycloalkyl, aryl, or heteroaryl;
W is O; and
V is O, N or S;
or salts thereof.

19. The compound of claim 18, wherein:
R₁ is t-BOC or acetyl;
R₂ is heterocycloalkyl;
R₃ is C(O)OR⁷, wherein R⁷ and R^{7′} are independently selected from alkyl, cycloalkyl, and aryl;
R₄ is H or alkyl;
R₅ and R₆ are independently H or alkyl;
A is COOH or COOR⁸, wherein R⁸ is alkyl, aryl, or heteroaryl;
W is O; and
V is O.

20. The compound of claim 18, wherein:
R₁ is t-BOC;
R₂ is isoindoline;
R₃ is C(O)OR⁷, wherein R⁷ is cycloalkyl;
R₄ is H;
R₅ and R₆ are H;
A is COOR⁸, wherein R⁸ is alkyl;
W is O; and
V is O.
